(19) United States
(12) Patent Application Publication
(10) Pub. No.: US 2017/0367875 A1
(43) Pub. Date: Dec. 28, 2017

(54) NOVEL CONDOM COMPRISING CANNABIS DERIVED COMPOSITIONS FOR ENHANCEMENT OF SEXUAL PLEASURE AND DECREASE OF ERECTILE DYSFUNCTION SYMPTOMS

(71) Applicant: One World Cannabis LTD, Petach Tikva (IL)

(72) Inventors: Alon Sinai, Petach Tikva (IL); Ziv Turner, Petach Tikva (IL); Yehuda Baruch, Gedera (IL)

(73) Assignee: One World Cannabis LTD, Petach Tikva (IL)

(21) Appl. No.: 15/536,738

(22) PCT Filed: Dec. 16, 2015
(86) PCT No.: PCT/IL2015/051220

§ 371 Date: Jun. 16, 2017

Related U.S. Application Data
(60) Provisional application No. 62/092,849, filed on Dec. 17, 2014.

(51) Int. Cl. A61F 6/04; A61K 31/352; A61K 9/00

(52) U.S. Cl. CPC A61F 6/04; A61K 9/0034; A61K 31/352

(57) ABSTRACT

The present invention provides a biocompatible polymer or biocompatible material or composition for forming at least part of the structure of a condom useful for preventing STI's. The biocompatible polymer incorporates cannabis or cannabis derived compositions. The present invention also provides a biocompatible additive for condoms incorporating cannabis or cannabis derived compositions in a carrier or excipient. In addition, the biocompatible polymer or biocompatible additive incorporating cannabis or cannabis derived compositions enhances pleasure during the sexual act. Lastly, the biocompatible polymer or biocompatible additive incorporating cannabis or cannabis derived compositions can be used for the treatment of erectile dysfunction.

### FIELD OF THE INVENTION

The present invention is in the field of contraceptive and prophylactic devices. More particularly, it pertains to providing condoms made of novel materials comprising cannabis or cannabis derived compositions for enhancement of sexual pleasure and decrease of erectile dysfunction symptoms, and to condoms additives (like lubricants) comprising cannabis or cannabis derived compositions for treatment of the same.

### BACKGROUND OF THE INVENTION

The use of a very wide variety of single use or multiple use condoms is ubiquitous.

A condom is a barrier device commonly used during sexual intercourse to reduce the probability of pregnancy and spreading sexually transmitted diseases. It is put on a man's erect penis and physically blocks ejaculated semen from entering the body of a sexual partner. Condoms are also used for collection of semen for use in infertility treatment. In the modern age, condoms are most often made from latex, but some are made from other materials such as polyurethane, polyisoprene, or lamb intestine. A female condom is also available, often made of nitrile. Condoms and female condoms only provide protection when used properly as a barrier, and only to and from the area that it covers. A research conducted by Osterger et. al., on a non-use condoms population, showed that almost 50% of the population didn't want to use condoms due to the feeling, or less feeling, that condoms provide (Jenny E. Ostergren, B. R. Simon Rosser, and Keith J. Horvath, Reasons for Non-use of Condoms among Men-who-have-Sex-with-Men: A Comparison of Receptive and Insertive Role-in-Sex and Online and Offline Meeting Venue, Cult Health Sex. February 2011; 13(2): 123-140—which is incorporated herein as reference).

Worldwide Definition of Sexual Pleasure: Sexuality can be inscribed in a multidimensional model comprising different aspects of human life: biology, reproduction, culture, entertainment, relationships and love. In the last decades, a growing interest towards sexuality and a greater quest to acknowledge a “right to sexuality” has occurred both in society and individuals. The consequence of this evolution has been a renewed and more explicit call for intervention from those who suffer, or think they suffer from alterations of their sexual and relational sphere.

This has produced an increased attention of medicine and psychology towards sexual dysfunctions and the problems they cause in individuals and couples. Science has gradually adjusted already existing research tools, mostly used in other fields of clinical research, to the field of sexology, so completing and increasing the number of tools in the “toolkit” of various branches of sexological diagnosis (http://en.wikipedia.org/wiki/Sexological_testing#ASEX_.28Arizona_Sexual_Experience_Scale.29, which is incorporated herein as reference).

Psychological measurements cannot be considered as accurate as physical ones (weight, height, mass, etc.), as the former evaluate those aspects and variables pertaining to an “individual” whose individuality refers to his/her own psychological, personological and environmental constituents: emotions, expressiveness, senses, feelings and experiences which can greatly vary according to the subjects and change in the short period or depending on different settings, even in the same individual.

What is expected of psychological measurements is “sufficient” accuracy and reliability, i.e. capability to express an indication or focus which clinicians can use as a “guideline” to rapidly and accurately deepen the aspects highlighted by the measurements and check them together with their patients. For this purpose, several statistical validation indexes of psychodiagnostic tests are provided: from standardization to various constructions of validity (internal, external, face, construct, convergent, content, discriminant, etc.).

There are several sexual dysfunctions and each of them has a different cause. Therefore, the field of sexology provides different psychological evaluation devices in order to examine the various aspects of the discomfort, problem or dysfunction, regardless of whether they are individual or relational ones.

The number of psychodiagnostic reactives is certainly wide and heterogeneous, nevertheless, the amount of tests specifically meant for the field of sexology is quite limited. The following list (in alphabetical order) is not exhaustive but shows the best known and/or most used reactives in the field of sexological and relational psychodiagnosis: ASEX (Arizona Sexual Experience Scale); ASKAS (Aging Sexuality Knowledge and Attitudes Scale); BSRI (Bem Sex Role Inventory); DAS (Dyadic Adjustment Scale); DIQ (Diagnostic Impotence Questionnaire); DSFI (Derogatis Sexual Function Inventory); EDITS (Erectile Dysfunction Inventory of Treatment Satisfaction); EPES (Erotic Preferences Examination Scheme); FACES (Family Adaptability and Cohesion Evaluation Scales); FGIS (Feminine Gender Identity Scale); GRIMS (Golombok Rust Inventory of Marital State); GRISS (Golombok Rust Inventory of Sexual Satisfaction); HSAS (Hendrick Sexual Attitude Scale); IIEF (International Index of Erectile Function); ISS (Index of Sexual Satisfaction); MAT (Marital Adjustment Test); MCI (Marital Communication Inventory); MMPI-2 (Minnesota Multiphasic Personality Inventory); MPT (Marital Patterns Test); MSI (Marital Satisfaction Inventory); PEQUEST (Premature Ejaculation Questionnaire); PREPARE-ENRICH (Premarital Personal and Relationship Evaluation); SAI (Sexual Arousability Inventory); SAS (Sexual Attitude Scale); SBI (Sexual Behavior Inventory); SESAMO_Win (Sexrelation Evaluation Schedule Assessment Monitoring on Windows); SESII-W (Sexual Excitation/Sexual Inhibition Inventory for Women); SFQ (Sexual Functioning Questionnaire); SHQ-R (Clarke Sex History Questionnaire for Males-Revised); SII (Sexual Interaction Inventory); SOC (Spouse Observation Checklist); SOS (Sexual Opinion Survey); TIPE (Test di Induzione Psico Erotica); and WIQ (Waring Intimacy Questionnaire).

These psychodiagnostic reactives are well accepted all over the world, and they confer a scale that can be used to identify in a quantitative matter the differences between two individual and independent experiences.

Functional magnetic resonance imaging or functional MRI (fMRI) is a functional neuroimaging procedure using MRI technology that measures brain activity by detecting associated changes in blood flow. This technique relies on the fact that cerebral blood flow and neuronal activation are coupled. When an area of the brain is in use, blood flow to that region also increases. The primary form of fMRI uses the blood-oxygen-level dependent (BOLD) contrast, discovered by Seiji Ogawa. This is a type of specialized brain and body scan used to map neural activity in the brain or spinal cord of humans or other animals by imaging the change in blood flow (hemodynamic response) related to energy use by brain cells. Since the early 1990s, fMRI has come to dominate brain mapping research because it does not require people to undergo shots, surgery, or to ingest substances, or be exposed to radiation, etc. Other methods of obtaining contrast are arterial spin labeling and diffusion MRI.

The procedure is similar to MRI but uses the change in magnetization between oxygen-rich and oxygen-poor blood as its basic measure. This measure is frequently corrupted by noise from various sources and hence statistical procedures are used to extract the underlying signal. The resulting brain activation can be presented graphically by color-coding the strength of activation across the brain or the specific region studied. The technique can localize activity to within millimeters but, using standard techniques, no better than within a window of a few seconds. fMRI is used both in the research world, and to a lesser extent, in the clinical world. It can also be combined and complemented with other measures of brain physiology such as EEG and NIRS. Newer methods which improve both spatial and time resolution are being researched, and these largely use biomarkers other than the BOLD signal. Some companies have developed commercial products such as lie detectors based on fMRI techniques, but the research is not believed to be ripe enough for widespread commercialization (http://en.wikipedia.org/wiki/Functional_magnetic_resonance_imaging, which is incorporated herein as reference).

fMRI, Pleasure and Pain: Most of what is known about pain and pleasure derives from the study of each phenomenon in isolation. Recently, however, neuroscientists investigating opioid and placebo analgesia, drug addiction and learning have begun to bridge the gap between the pain and pleasure research fields. This development has been strengthened by the increasing focus on the subjective emotional feelings (hedonics) that are elicited by rewards and punishments. Rewards and punishments are defined as something that an animal will work to achieve or avoid, respectively. Pleasure represents the subjective hedonic value of rewards. The term ‘pain’ encompasses both the hedonic (suffering) and motivational (avoidance) aspects of a painful experience. Clearly, seeking pleasure and avoiding pain is important for survival, and these two motivations probably compete for preference in the brain. Put simply, which of two coinciding pain and pleasure events should be processed and acted on first? Consistent with the idea that a common currency of emotion enables the comparison of pain and pleasure in the brain, the evidence reviewed points to there being extensive overlap in the neural circuitry and chemistry of pain and pleasure processing at the systems level (Leknes and Tracey, “A common neurobiology for pain and pleasure”, Nature Reviews Neuroscience 9, 314-320 (April 2008)—which is incorporated herein as reference). As shown in this scientific article, the brain pathways that are involved during pleasure and pain can be mapped and defined by functional MRI (fMRI). This mapping system provides a measurable and scalable feedback of the level of pleasure. Which, together with the aforementioned psychodiagnostic reactives, provide the tools for a concrete measure of pleasure.

Erectile dysfunction (ED) or impotence is sexual dysfunction characterized by the inability to develop or maintain an erection of the penis during sexual activity. A penile erection is the hydraulic effect of blood entering and being retained in sponge-like bodies within the penis. The process is often initiated as a result of sexual arousal, when signals are transmitted from the brain to nerves in the penis. The most important organic causes are cardiovascular disease and diabetes, neurological problems (for example, trauma from prostatectomy surgery), hormonal insufficiencies (hypogonadism) and drug side effects (http://en.wikipedia.org/wiki/Erectile_dysfunction, which is incorporated herein as reference).

Psychological impotence is where erection or penetration fails due to thoughts or feelings (psychological reasons) rather than physical impossibility; this is somewhat less frequent but can often be helped. Notably in psychological impotence, there is a strong response to placebo treatment. Erectile dysfunction can have severe psychological consequences as it can be tied to relationship difficulties and masculine self-image generally.

Besides treating the underlying causes such as potassium deficiency or arsenic contamination of drinking water, the first line treatment of erectile dysfunction consists of a trial of PDE5 inhibitor drugs (the first of which was sildenafil or Viagra). In some cases, treatment can involve prostaglandin tablets in the urethra, injections into the penis, a penile prosthesis, a penis pump or vascular reconstructive surgery.

The Latin term impotentia coeundi describes simple inability to insert the penis into the vagina; it is now mostly replaced by more precise terms, such as erectile dysfunction (ED). The study of erectile dysfunction within medicine is covered by andrology, a sub-field within urology. Research indicates that erectile dysfunction is common, and it is suggested that approximately 40% of males suffer from erectile dysfunction or impotence, at least occasionally.

Medical cannabis (or medical marijuana) refers to the use of cannabis and its constituent cannabinoids, such as tetrahydrocannabinol (THC) and cannabidiol (CBD), as medical therapy to treat disease or alleviate symptoms. The Cannabis plant has a history of medicinal use dating back thousands of years across many cultures. Its usage in modern times is controversial, and in recent years the American Medical Association, the MMA, the American Society of Addiction Medicine, and other medical organizations have issued statements opposing its usage for medicinal purposes (http://cn.wikipedia.org/wiki/Medical_cannabis, which is incorporated herein as reference).

Medical cannabis can be administered using a variety of methods, including vaporizing or smoking dried buds, eating extracts, taking capsules or using oral sprays. Synthetic cannabinoids are available as prescription drugs in some countries; examples include: dronabinol (available in the United States (US) and Canada) and nabilone (available in Canada, Mexico, the United Kingdom (UK), and the US). Recreational use of cannabis is illegal in most parts of the world, but the medical use of cannabis is legal in certain countries, including Austria, Canada, Czech Republic, Finland, Germany, Israel, Italy, the Netherlands, Portugal and Spain. In the US, federal law outlaws all cannabis use, while 20 states and the District of Columbia no longer prosecute individuals merely for the possession or sale of marijuana, as long as the individuals are in compliance with the state's marijuana sale regulations. However, an appeals court ruled in January 2014 that a 2007 Ninth Circuit ruling remains binding in relation to the ongoing illegality, in federal legislative terms, of Californian cannabis dispensaries, reaffirming the impact of the federal Controlled Substances Act.

Regarding the method of delivery, controlled release medical compositions and pharmaceuticals have become very important in the treatment of many medical conditions. Controlled release dosage forms tend to maintain more consistent blood serum levels with less fluctuation, and thus may reduce undesirable side effects. However, because of the nature of certain types of drugs, often, it is desirable to modify or carry drugs in specific ways for even immediate release drugs. Thus, improved controlled release formulations and immediate release formulations that provide certain advantages continue to be sought.

Thus it would be very useful to have a treatment or therapy that effectively improves or cures erectile dysfunction while protecting the subject from STI's.

Several studies have been done regarding the effects of cannabis. As presented in the scientific report published in Clinical and Developmental Immunology, written by Rodrigo Araujo Fraga-Silva et al. in their publication titled “Treatment with CB2 Agonist JWH-133 Reduces Histological Features Associated with Erectile Dysfunction in Hypercholesterolemic Mice” (which is incorporated herein as reference), cannabis may be a strong tool to treat erectile dysfunction.

There is a longstanding, long felt need to provide improved condoms with multiple purposes. On one side, protection from STI's including HIV and Hepatitis (when spread by sexual contact), and on the other side, the ability of not to lose pleasure during coitus because of the usage of condoms. In addition, at the same time, providing an efficient treatment for erectile dysfunction.

### SUMMARY OF THE INVENTION

Thus, it is an object of the present invention to provide a biocompatible polymer or biocompatible material or composition for forming at least part of the structure of a condom useful for preventing STI's. The biocompatible polymer incorporates cannabis or cannabis derived compositions. Another object of the present invention is to provide a biocompatible additive for condoms incorporating cannabis or cannabis derived compositions in a carrier or excipient. In addition, the biocompatible polymer or biocompatible additive incorporating cannabis or cannabis derived compositions enhances pleasure during the sexual act. Lastly, the biocompatible polymer or biocompatible additive incorporating cannabis or cannabis derived compositions can be used for the treatment of erectile dysfunction.

A condom for providing increased protection from STI's including HIV and Hepatitis comprising a biocompatible material or composition for forming at least part of the structure of a condom, wherein said biocompatible material or composition incorporates cannabis or cannabis derived compositions; and further wherein said condom is adapted to increase the pleasure felt when compared to the pleasure felt when performing the same action but in absence of the present invention, measured using standard scales determined in sexology psychodiagnostic reactives.

It is an object of the present invention to provide a condom for providing increased protection from STI's including HIV and Hepatitis comprising a biocompatible material or composition for forming at least part of the structure of a condom, where said biocompatible material or composition incorporates cannabis or cannabis derived compositions; and further wherein said condom is adapted to increase the pleasure felt when compared to the pleasure felt when performing the same action but in absence of the present invention, measured using functional MRI methods.

It is further an object of the present invention to provide a condom where said condom is substantially made of latex.

It is further an object of the present invention to provide a condom where said condom is substantially made of a material selected from the group consisting of latex, polyurethane, polyisoprene, nitrile, lamb intestine or any combination thereof.

It is further an object of the present invention to provide a condom where said condom is a male condom.

It is further an object of the present invention to provide a condom where said condom is a female condom.

It is further an object of the present invention to provide a condom where said condom complies with essential performance attributes of ISO 4074 Natural Latex condoms requirements and test methods.

It is further an object of the present invention to provide a condom where said biocompatible material complies with ISO 10993-1.

It is further an object of the present invention to provide a condom where the cytotoxicity of said biocompatible material complies with ISO 10993-5.

It is further an object of the present invention to provide a condom where the irritation and sensitization values of said biocompatible material complies with ISO 10993-10.

It is further an object of the present invention to provide a condom where the water extractable level for soluble proteins is less than 200 ug/g as measured by the Lowry method.

It is further an object of the present invention to provide a condom where said cannabis or cannabis derived compositions adapted to provide activation of neurological pathways during use thereby enhancing sensation.

It is further an object of the present invention to provide a condom where the lubricant of said condom comprises cannabis or cannabis derived compositions.

It is further an object of the present invention to provide a condom where said lubricant of said condom additionally comprises silicone fluid.

It is further an object of the present invention to provide a condom where said lubricant of said condom additionally comprises glycol or a water based lubricant.

It is further an object of the present invention to provide a condom where said cannabis or cannabis derived compositions can be applied to said condom on the inside, the outside or both.

It is further an object of the present invention to provide a condom where said condom additionally comprises nonoxynol-9 as a spermicide.

It is further an object of the present invention to provide a condom where said condom additionally comprises a bactericide, viricide or spermicide or any combination thereof.

It is an object of the present invention to provide a biocompatible material or composition for forming at least part of the structure of a condom, wherein said biocompatible material or composition incorporates cannabis or cannabis derived compositions and provides enhanced protection from STI's when compared with said biocompatible material without said incorporation of cannabis or cannabis derived compositions.

It is further an object of the present invention to provide a biocompatible material where the biocompatible material or composition adapted for ex tempora application as a cream, gel or foam on any conventional condom.

It is further an object of the present invention to provide a condom for treating erectile dysfunction disorder comprising a biocompatible material or composition for forming at least part of the structure of a condom, wherein said biocompatible material or composition incorporates cannabis or cannabis derived compositions.

It is further an object of the present invention to provide a condom further comprising an additive comprising (a) cannabis or cannabis derived compositions, (b) at least one pharmaceutically acceptable carrier or excipient, wherein said composition is adapted to be penile or vaginally administrable further wherein said composition is in an immediate release form.

It is further an object of the present invention to provide a condom where said carrier or excipient is selected from a group consisting of diluents, binders, granulating agents, glidants, lubricants, surfactants, disintegrates, dissolving agents, solubilising agents, bioadhesive agents, polysaccharides, polymers, copolymers, bioavailability enhancing agent, acidifying agents, probiotic agents, protective agents, antioxidants, dispersing agents and any combination thereof.

It is further an object of the present invention to provide a condom where said composition has disintegration time of between about 3 minutes and about 10 minutes in the penis or vagina.

It is further an object of the present invention to provide a condom where said additive can be applied to the inside, the outside or both.

It is further an object of the present invention to provide a condom for providing increased protection from STI's including HIV and Hepatitis, further providing enhanced pleasure, comprising an additive comprising (a) cannabis or cannabis derived compositions, (b) at least one pharmaceutically acceptable carrier or excipient, wherein said additive is adapted to be penile or vaginally administrable further wherein said additive is in an immediate release form, prepared by steps of: preparing a mixture comprising (i) an effective amount of at least one active ingredient selected from a group consisting of cannabis or cannabis derived compositions, and (ii) at least one pharmaceutically acceptable carrier or excipient and, forming said additive in an immediate release form; applying said additive to said condom.

It is further an object of the present invention to provide an additive for penile or vaginal administration prepared by said steps, where said mixture additionally comprises a lubricating agent.

It is further an object of the present invention to provide an additive for penile or vaginal administration prepared by said steps, where said composition is further prepared by steps of incorporating into said mixture at least one carrier or excipient selected from a group consisting of diluents, binders, granulating agents, glidants, lubricants, surfactants, disintegrates, dissolving agents, solubilising agents, bioadhesive agents, polysaccharides, polymers, copolymers, bioavailability enhancing agent, acidifying agents, probiotic agents, protective agents, antioxidants, dispersing agents, add-on” formulation excipients and any combination thereof.

It is further an object of the present invention to provide the use of a penile or vaginally administrable additive, for the manufacture of a pleasure enhancer condom, said additive comprising (a) cannabis or cannabis derived compositions, (b) at least one pharmaceutically acceptable carrier or excipient, wherein said additive is adapted to be penile or vaginally administrable further wherein said additive is in an immediate release form.

It is further an object of the present invention to provide the use of a penile or vaginally administrable additive, for the manufacture of a pleasure enhancer condom where said composition is further prepared by steps of incorporating into said mixture at least one carrier or excipient selected from a group consisting of diluents, binders, granulating agents, glidants, lubricants, surfactants, disintegrates, dissolving agents, solubilizing agents, bioadhesive agents, polysaccharides, polymers, copolymers, bioavailability enhancing agent, acidifying agents, probiotic agents, protective agents, antioxidants, dispersing agents, add-on” formulation excipients and any combination thereof.

It is further an object of the present invention to provide the use of a penile or vaginally administrable additive, for the manufacture of a medicament for the treatment of erectile dysfunction disorder, said additive comprising (a) cannabis or cannabis derived compositions, (b) at least one pharmaceutically acceptable carrier or excipient, wherein said additive is adapted to be penile or vaginally administrable further wherein said additive is in an immediate release form.

It is further an object of the present invention to provide the use of a penile or vaginally administrable additive, for the manufacture of a medicament for the treatment of erectile dysfunction disorder, where said composition is further prepared by steps of incorporating into said mixture at least one carrier or excipient selected from a group consisting of diluents, binders, granulating agents, glidants, lubricants, surfactants, disintegrates, dissolving agents, solubilizing agents, bioadhesive agents, polysaccharides, polymers, copolymers, bioavailability enhancing agent, acidifying agents, probiotic agents, protective agents, antioxidants, dispersing agents, add-on” formulation excipients and any combination thereof.

### BRIEF DESCRIPTION OF THE DRAWINGS

FIG. 1 illustrates a condom of the type used in the present invention, prior to unrolling.

FIG. 2 illustrates a condom of the type used in the present invention, after unrolling.

FIG. 3 illustrates three possible embodiments of the present invention.

### DETAIL DESCRIPTION OF THE PREFERRED EMBODIMENTS

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide condoms made of novel materials comprising cannabis derived compositions and/or to provide condoms comprising additives made of materials comprising cannabis derived compositions for the enhancement of pleasure and for treatment of erectile dysfunction.

It is herein acknowledged that some embodiments of the present invention prevent Sexual Transmitted Infections (STI's) due to the functional properties of the condom per se, while at the same time enhancement of pleasure can be successfully achieved by the addition of cannabis derived compositions incorporated in the materials of the condom or as additives to the already manufactured condom. Furthermore, erectile dysfunction pathologies can be effectively treated by the same way.

It is herein acknowledged that some embodiments of the present invention provide better erections due to chemical properties of cannabis or cannabis derived compositions incorporated in the materials of the condom or as additives to the already manufactured condom.

It is herein acknowledged that some embodiments of the present invention provide enhanced sensation during use due to chemical properties of cannabis or cannabis derived compositions incorporated in the materials of the condom or as additives to the already manufactured condom.

The condom is also provided wherein the incorporated cannabis or cannabis derived composition is adapted to provide activation of neurological pathways during use thereby enhancing sensation.

The enhancement of sensation and thereby pleasure, can be measured by the neurological pathways activated during the usage of the present invention and compared to the neurological pathways activated during the usage of a similar product lacking the cannabis or cannabis derived compositions.

It is herein acknowledged that the cannabis or cannabis derived compositions may be incorporated within or applied as a coating or coatings to any material suitable for a condom, whether it be an already acknowledged material or a novel material which will be rendered suitable for condom use as herein described by the application of cannabis or cannabis derived compositions.

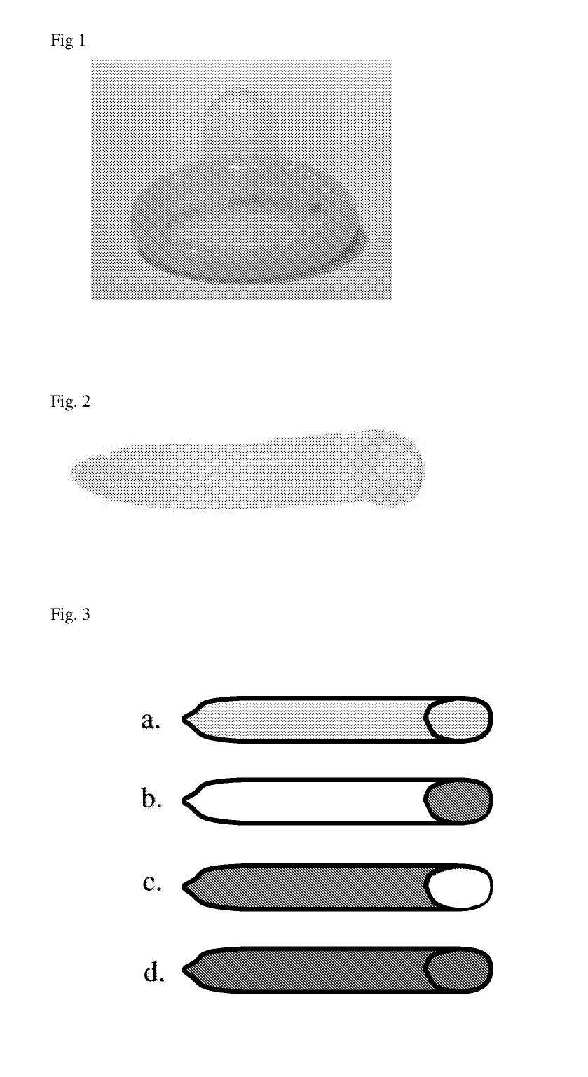

With respect to the present invention, FIGS. 1 and 2 show conventional condoms in their rolled and unrolled state. It is a core purpose of the present invention to provide condoms of similar shape enhanced with biocompatible polymers for forming at least part of the structure or as an additive coating of male of female condom, said biocompatible polymers incorporating cannabis or cannabis derived compositions.

It is herein acknowledged that the present invention provides biocompatible materials incorporating cannabis or cannabis derived compositions for use in or on condoms, such biocompatible materials being useful for enhancing pleasure and/or also for treating erectile dysfunction disorders. The condoms comprise biocompatible materials selected from the group consisting of polymeric, hydrophobic, hydrophilic, soft or multicomponent or multi-textural or any combination thereof.

It is herein acknowledged that the present invention provides biocompatible materials incorporating cannabis or cannabis derived compositions with bactericidal, bacteriostatic and/or antiviral properties for use in condoms.

It is herein acknowledged that the present invention provides biocompatible materials incorporating cannabis or cannabis derived compositions used in condoms with minimal or reduced allergic response or reaction.

With respect to the present invention, FIG. 3a show an illustration of a condom that is made from biocompatible materials incorporating cannabis or cannabis derived compositions.

It is herein acknowledged that the present invention provides biocompatible materials for use in medical devices, such biocompatible materials compliant with any or the following standards:

Good manufacturing practices (GMP) rules are standards for factories that make drugs, including products like condoms and hand sanitizers that play a role in preventing disease. In the United States, GMP rules fall under the jurisdiction of the Food and Drug Administration (FDA). Internationally, the International Organization for Standardization has created a standard called ISO 9000. Another ISO standard, ISO 13485, covers medical devices and is used in many areas to regulate condom production. These sets of rules cover everything from manufacturing methods to record keeping in ways that can apply to all drugs. What they do not do is govern the specifications of the condoms that leave the factory. Specific condom standards are therefore widely recommended and used.

While many countries have their own standards, at least two international standards set guidelines for everything from how condoms are tested to what color they are. The primary international standard is ISO 4074:2002. The World Health Organization (WHO) Male Latex Condom Specification uses ISO standards as a foundation for its guidelines on purchasing condoms for health promotion.

The ISO and WHO specifications for condoms include parameters for:
Acceptable quality levels (AQLs), or the maximum number of condoms that can be defective in each batch
Accreditation for laboratories that test condoms
Procedures for the tests
Materials, shelf life and stability

The ISO and WHO standards also outline passing and failing grades for the tests described in Zapping, Popping, Rolling and Other Condom Testing Tools.

It is herein acknowledged that the present invention provides at least part of the structure of condom incorporating or coated with or impregnated with a biocompatible polymer or material incorporating cannabis or cannabis derived compositions.

Reference is now made to the core of the present invention, namely a condom for providing increased protection from STI's including HIV and Hepatitis comprising a biocompatible material or composition for forming at least part of the structure of a condom, wherein said biocompatible material or composition incorporates cannabis or cannabis derived compositions.

Reference is now made to an aspect of the present invention namely the abovementioned condom wherein said condom is substantially made of latex.

Reference is now made to another aspect of the present invention wherein said condom is substantially made of a material selected from the group consisting of latex, polyurethane, polyisoprene, nitrile, lamb intestine or any combination thereof.

Reference is now made to another aspect of the present invention wherein said condom is a male condom.

Reference is now made to another aspect of the present invention wherein said condom is a female condom.

Reference is now made to another aspect of the present invention wherein said condom complies with essential performance attributes of ISO 4074:2002 Natural Latex condoms requirements and test methods.

Reference is now made to another aspect of the present invention wherein said biocompatible material complies with ISO 10993-1.

Reference is now made to another aspect of the present invention wherein the cytotoxicity of said biocompatible material complies with ISO 10993-5.

Reference is now made to another aspect of the present invention wherein the irritation and sensitization values of said biocompatible material complies with ISO 10993-10.

Reference is now made to another aspect of the present invention wherein the water extractable level for soluble proteins is less than 200 ug/g as measured by the Lowry method.

Reference is now made to another aspect of the present invention wherein the lubricant of said condom comprises cannabis or cannabis derived compositions.

Reference is now made to another aspect of the present invention wherein said lubricant of said condom additionally comprises silicone fluid.

Reference is now made to another aspect of the present invention wherein said lubricant of said condom additionally comprises glycol or a water based lubricant.

Reference is now made to another aspect of the present invention wherein said condom additionally comprises nonoxynol-9 as a spermicide.

Reference is now made to another aspect of the present invention wherein said condom additionally comprises a bacteriocide, viricide or spermicide or any combination thereof.

Reference is now made to a biocompatible material or composition for forming at least part of the structure of a condom, wherein said biocompatible material or composition incorporates cannabis or cannabis derived compositions and provides enhanced protection from STI's when compared with said biocompatible material or composition without said incorporation cannabis or cannabis derived compositions. Reference is now made to another aspect of the present invention wherein said biocompatible material or composition is adapted as a cream, gel or foam for ex tempora application on any conventional condom.

The present invention provides cannabis or cannabis derived compositions useful for treating erectile dysfunction. The composition comprises cannabis or cannabis derived active ingredients and at least one pharmaceutically acceptable carrier or excipient. It is a core aspect of the invention to provide the aforementioned composition adapted to be administrable via the skin of either the penis or the vagina. In a further core aspect the composition is in an immediate release form.

Penile administration has several advantages. The penile area is characterized by a rich blood supply, resulting in rapid and steady uptake of drugs, lower serum concentrations than oral drug delivery and a decrease in first-pass liver metabolism, which allows low doses with fewer side effects.

By using penile administration, the same effect is obtained with much lower serum concentrations of the therapeutic agent. A higher penile concentrations of the active ingredient is achieved after penile than after oral administration.

The present invention further provides a composition for penile administration with improved bioavailability, bioadhesiveness, disintegrating and solubility properties and rapid release and uptake of the drugs in the affected penile tissue to effectively treat erectile dysfunction disorders.

In a certain embodiment, formulations can be configured as a coat layer upon a substrate such as a condom adapted for penile or vaginal usage. These configurations are herein referred to as “add-on” formulations.

As used herein the term “bioavailability” refers to a pharmacokinetic property which is used to describe the fraction of an administered dose of a drug that reaches the systemic circulation. It is herein acknowledged, that when a medication is administered intravenously, its bioavailability is defined as 100%. However, when a medication is administered via other routes (such as orally), its bioavailability decreases.

The term “absolute bioavailability” used herein refers to the bioavailability of an active drug in systemic circulation or bloodstream following non-intravenous administration (i.e., after oral, rectal, transdermal, subcutaneous, vaginal or sublingual administration), relative to the bioavailability of the same drug following intravenous administration. In pharmacology, the absolute bioavailability of a drug is determined by pharmacokinetic parameters defining the plasma drug concentration versus time plot for the drug after both intravenous (IV) and non-intravenous administration. The absolute bioavailability may be defined as the dose-corrected area under curve (AUC) obtained by non-intravenous divided by AUC obtained by intravenous administration. For example, the formula for calculating the bioavailability (F) for a drug administered by the oral route (po) is given below:

$$F = \frac{{[ AUC ]}_{po} * {dose}_{IV}}{{[ AUC ]}_{IV} * {dose}_{po}}$$

According to some embodiments of the present invention, the absolute bioavailability of the composition refers to the fraction of the drug absorbed through penile or vaginal administration compared with the corresponding intravenous administration of the same drug. It is within the scope of the present invention that this ratio is compared to the ratio obtained by oral application of cannabis or cannabis derived compositions.

The term “relative bioavailability” used herein refers to the bioavailability (estimated as the AUC defined above) of a certain drug relative to the bioavailability of a different formulation of the same drug, for example the same drug formulated for administration via a different route. When the relative bioavailability is measured as compared to an intravenously administered drug, the following formula may be used:

$$relative bioavailability = \frac{{[ AUC ]}_{A} * {dose}_{B}}{{[ AUC ]}_{B} * {dose}_{A}}$$

The term ‘immediate release’ used herein refers to the properties of the penile administrable composition, having a disintegration time of less than about 3 minutes in water at room temperature or less than about 10 minutes in the penis.

In accordance with a further embodiment of the invention, the penile administrable composition comprises at least one pharmaceutically acceptable non-effervescent excipient, polymer, copolymer and/or carrier. The excipients may include diluents, binders, surfactants, polymers, copolymers, polysaccharides or granulating agents, glidants (flow aids) and lubricants; and, disintegrants. A polymer coating is often applied to make the carrier smoother, to control the release rate of the active ingredient, to make it more resistant to the environment (extending its shelf life), and/or to enhance the carrier's appearance. In addition such excipients may include pigments to make the carrier visually attractive.

In accordance with a further embodiment of the invention, the non-effervescent excipient is further selected from a group comprising Hydroxypropyl methyl cellulose (HPMC), starch, kollidone, ethanol, Klucel, Ethocel, FMC nanoparticles, Carbopol, Lactose, Magnesium Stearat, Silicon dioxide, lipidic carriers, gums, cellulose derivatives and mixtures thereof.

With respect to the present invention, FIGS. 3b-d show illustration of different embodiments where: b. a condom with an additive incorporating cannabis or cannabis derived compositions on the inside only; c. a condom with an additive incorporating cannabis or cannabis derived compositions on the outside only; and d. a condom with an additive incorporating cannabis or cannabis derived compositions on the inside and on the outside. In this way, the present invention delivers the benefits of the cannabis product or additive or composition to both participants in the sexual act.

The present invention further provides a method for providing a condom for providing increased protection from STI's including HIV and Hepatitis comprising an additive comprising (a) cannabis or cannabis derived compositions, (b) at least one pharmaceutically acceptable carrier or excipient, where the additive is adapted to be penile or vaginally administrable further wherein said additive is in an immediate release form, prepared by steps of: preparing a mixture comprising (i) an effective amount of at least one active ingredient selected from a group consisting of cannabis or cannabis derived compositions, and (ii) at least one pharmaceutically acceptable carrier or excipient; forming said additive in an immediate release form; and applying said additive to said condom.

The additive for penile or vaginal administration prepared by steps according to the method as defined above, where said mixture additionally comprises a lubricating agent.

The additive for penile or vaginal administration prepared by steps according to the method as defined above, where said composition is further prepared by steps of incorporating into said mixture at least one carrier or excipient selected from a group consisting of diluents, binders, granulating agents, glidants, lubricants, surfactants, disintegrates, dissolving agents, solubilising agents, bioadhesive agents, polysaccharides, polymers, copolymers, bioavailability enhancing agent, acidifying agents, probiotic agents, protective agents, antioxidants, dispersing agents, add-on” formulation excipients and any combination thereof.

Reference is now made to a use of a penile or vaginally administrable additive, for the manufacture of a medicament for the treatment of erectile dysfunction disorder, said additive comprising (a) cannabis or cannabis derived compositions, (b) at least one pharmaceutically acceptable carrier or excipient, where said additive is adapted to be penile or vaginally administrable further wherein said additive is in an immediate release form.

The use according to the use as defined above, where said composition is further prepared by steps of incorporating into said mixture at least one carrier or excipient selected from a group consisting of diluents, binders, granulating agents, glidants, lubricants, surfactants, disintegrates, dissolving agents, solubilizing agents, bioadhesive agents, polysaccharides, polymers, copolymers, bioavailability enhancing agent, acidifying agents, probiotic agents, protective agents, antioxidants, dispersing agents, add-on” formulation excipients and any combination thereof.

It is a scope of the present invention to provide a condom for providing increased pleasure when compared to the pleasure felt when performing the same action but in absence of the present invention, measured using any of the scales determined in sexology psychodiagnostic reactives.

It is a scope of the present invention to provide a condom for providing increased pleasure when compared to the pleasure felt when performing the same action but in absence of the present invention, measured using any of the scales determined in functional MRI.

1.-32. (canceled)

33. A condom for providing increased sexual pleasure and protection from Sexual Transmitted Infections (STI's) including HIV and Hepatitis comprising a biocompatible material or composition for forming at least part of the structure of a condom, wherein said biocompatible material or composition incorporates cannabis or cannabis derived active ingredients in an immediate release form, having a predefined disintegration time of said active ingredient; further wherein said condom is adapted to increase the pleasure felt when compared to the pleasure felt when performing the same action but in absence of the present invention, measured using at least one of (a) standard scales determined in sexology psychodiagnostic reactives, and (b) functional MRI methods.

34. The condom according to claim 33, wherein said condom is characterized by at least one characteristic selected from the group consisting of:
a. substantially made of latex;
b. is substantially made of a material selected from the group consisting of latex, polyurethane, polyisoprene, nitrile, lamb intestine and any combination thereof;
c. is a male condom;
d. is a female condom;
e. said condom complies with essential performance attributes of ISO 4074 Natural Latex condoms requirements and test methods.

35. The condom of claim 33, wherein said biocompatible material is characterized by at least one characteristic selected from the group consisting of:
a. complies with ISO 10993-1;
b. the cytotoxicity of said biocompatible material complies with ISO 10993-5;
c. the irritation and sensitization values of said biocompatible material complies with ISO 10993-10.

36. The condom of claim 33, wherein the water extractable level for soluble proteins is less than 200 μg/g as measured by the Lowry method.

37. The condom of claim 33, wherein said cannabis or cannabis derived active ingredients are at least one of:
a. adapted to provide activation of neurological pathways during use thereby enhancing sensation;
b. can be applied to said condom on the inside, the outside or both.

38. The condom of claim 33, wherein said condom comprises lubricant and said lubricant comprises cannabis or cannabis derived active ingredients.

39. The condom of claim 38, wherein said lubricant of said condom additionally comprises at least one of:
a. silicone fluid;
b. glycol or a water based lubricant.

40. The condom of claim 33, wherein said condom additionally comprises at least one of:
a. nonoxynol-9 as a spermicide;
b. a bactericide, viricide or spermicide or any combination thereof.

41. A biocompatible material or composition for forming at least part of the structure of a condom, wherein said biocompatible material or composition incorporates cannabis or cannabis derived active ingredients in an immediate release form, having a predefined disintegration time of said active ingredient and provides enhanced protection from STI's when compared with said biocompatible material without said incorporation of cannabis or cannabis derived compositions.

42. The biocompatible material or composition of claim 41, wherein the biocompatible material or composition is adapted for ex tempora application as a cream, gel or foam on any conventional condom.

43. The condom according to claim 33, wherein said condom is useful for treating erectile dysfunction disorder.

44. The condom of claim 33, further comprising an additive comprising (a) cannabis or cannabis derived active ingredients, (b) at least one pharmaceutically acceptable carrier or excipient, wherein said composition is adapted to be penile or vaginally administrable further wherein said composition is in an immediate release form; further wherein said carrier or excipient is selected from a group consisting of diluents, binders, granulating agents, glidants, lubricants, surfactants, disintegrates, dissolving agents, solubilising agents, bioadhesive agents, polysaccharides, polymers, copolymers, bioavailability enhancing agent, acidifying agents, probiotic agents, protective agents, antioxidants, dispersing agents and any combination thereof.

45. The condom of claim 44, wherein said composition has disintegration time of between about 3 minutes and about 10 minutes in the penis or vagina.

46. The condom of claim 44, wherein said additive can be applied to said condom on the inside, the outside or both.

47. A condom for providing increased sexual pleasure and protection from STI's including HIV and Hepatitis, further providing enhanced pleasure, comprising an additive comprising (a) cannabis or cannabis derived compositions in an immediate release form, having a predefined disintegration time of the active ingredient, (b) at least one pharmaceutically acceptable carrier or excipient, wherein said additive is adapted to be penile or vaginally administrable further wherein said additive is in an immediate release form, prepared by steps of:
a. preparing a mixture comprising (i) an effective amount of at least one active ingredient selected from a group consisting of cannabis or cannabis derived compositions, and (ii) at least one pharmaceutically acceptable carrier or excipient;
b forming said additive in an immediate release form; and
c. applying said additive to said condom.

48. A condom prepared by steps according to claim 47, wherein said mixture additionally comprises a lubricating agent.

49. A condom prepared by steps according to claim 47, wherein said composition is further prepared by steps of incorporating into said mixture at least one carrier or excipient selected from a group consisting of diluents, binders, granulating agents, glidants, lubricants, surfactants, disintegrates, dissolving agents, solubilising agents, bioadhesive agents, polysaccharides, polymers, copolymers, bioavailability enhancing agent, acidifying agents, probiotic agents, protective agents, antioxidants, dispersing agents, add-on formulation excipients and any combination thereof.
